(19) United States
(12) Patent Application Publication
(10) Pub. No.: US 2025/0225274 A1
(43) Pub. Date: Jul. 10, 2025

(54) SYSTEMS AND METHODS FOR DATA NORMALIZATION, ALGORITHM CERTIFICATION AND REPORT GENERATION IN A TRUSTED COMPUTING ENVIRONMENT

(71) Applicant: BeeKeeperAI, Inc., Austin, TX (US)

(72) Inventors: Michael Scott Blum, Scottsdale, AZ (US); Mary Elizabeth Chalk, Austin, TX (US); Alan Donald Czeszynski, Pleasanton, CA (US); Sudish Mogli, San Jose, CA (US); Robert Derward Rogers, Oakland, CA (US)

(21) Appl. No.: 19/094,629

(22) Filed: Mar. 28, 2025

Related U.S. Application Data
(63) Continuation-in-part of application No. 18/168,560, filed on Feb. 13, 2023.
(60) Provisional application No. 63/313,774, filed on Feb. 25, 2022.

(51) Int. Cl. G06F 21/62; G06F 5/01; G06F 11/30

(52) U.S. Cl. CPC G06F 21/6245; G06F 5/01; G06F 11/3072

(57) ABSTRACT

Systems and methods related to normalization of runtime data for algorithm training in a zero-trust computing environment are provided. The data set is first projected into a feature space using at least one transform model, and used to extract identified features of a dataset, yielding a pre-processed dataset before subsequent runtime algorithm consumption. Systems and methods are also presented for algorithm certification in a zero-trust computing environment, and report generation in a zero-trust computing environment. In yet another embodiment, systems and methods for processing data using a foundational model for data curation are provided. These systems and methods enable more efficient algorithm deployment and operation.

### CROSS REFERENCE TO RELATED APPLICATIONS

This U.S. Application is a continuation-in-part of U.S. application Ser. No. 18/168,560, filed Feb. 13, 2023, which claims the benefit and priority of U.S. Application No. 63/313,774, filed Feb. 25, 2022, the contents of which are incorporated herein in their entirety by this reference.

### BACKGROUND

The present invention relates in general to the field of confidential computing, and more specifically to methods, computer programs and systems for data normalization for processing, certification of algorithms and report generation from algorithm processing of private datasets. Such systems and methods are particularly useful in for ensuring more accurate algorithm processing in a more secure (less prone to data leakage) manner, with the output of relevant and useful reporting information.

Within certain fields, there is a distinguishment between the developers of algorithms (often machine learning of artificial intelligence algorithms), and the stewards of the data that said algorithms are intended to operate with and be trained by. For the avoidance of doubt, an algorithm may include a model, code, pseudo-code, source code, or the like. On its surface, this seems to be an easily solved problem of merely sharing either the algorithm or the data that it is intended to operate with. However, in reality, there is often a strong need to keep the data and the algorithm secret. For example, the companies developing their algorithms may have the bulk of their intellectual property tied into the software comprising the algorithm. For many of these companies, their entire value may be centered in their proprietary algorithms. Sharing such sensitive data is a real risk to these companies, as the leakage of the software base code could eliminate their competitive advantage overnight.

One could imagine that instead, the data could be provided to the algorithm developer for running their proprietary algorithms and generation of the attendant reports. However, the problem with this methodology is two-fold. Firstly, often the datasets for processing are often extremely large, requiring significant time to transfer the data from the data steward to the algorithm developer. Indeed, sometimes the datasets involved consume petabytes of data. The fastest fiber optics internet speed in the US is 2,000 MB/second. At this speed, transferring a petabyte of data can take nearly seven days to complete. It should be noted that most commercial internet speeds are a fraction of this maximum fiber optic speed.

The second reason that the datasets are not readily shared with the algorithm developers is that the data itself may be secret in some manner. For example, the data could also be proprietary, being of a significant asset value. Moreover, the data may be subject to some controls or regulations. This is particularly true in the case of medical information. Protected health information, or PHI, for example, is subject to a myriad of laws, such as HIPAA and GDPR, that include strict requirements on the sharing of PHI, and are subject to significant fines if such requirements are not adhered to.

Healthcare related information is of particular focus in this application. Of all the global stored data, about 30% resides in healthcare. This data provides a treasure trove of information for algorithm developers to train their specific algorithm models (AI or otherwise) and allows for the identification of correlations and associations within datasets. Such data processing allows advancements in the identification of individual pathologies, public health trends, treatment success metrics, and the like. Such output data from the running of these algorithms may be invaluable to individual clinicians, healthcare institutions, and private companies (such as pharmaceutical and biotechnology companies). At the same time, the adoption of clinical AI has been slow. Data access is a major barrier to clinical approval. The FDA requires proof that a model works across the entire population. However, privacy protections make it challenging to access enough diverse data to accomplish this goal.

Given that there is great value in the ability to operate models in an efficient and accurate manner in a secure way, systems and methods of enabling such algorithm operations are highly sought after.

### SUMMARY

The present systems and methods relate to normalization of runtime data for algorithm training in a zero-trust computing environment. Systems and methods are also presented for algorithm certification in a zero-trust computing environment, and report generation in a zero-trust computing environment. These systems and methods enable more efficient algorithm deployment and operation.

For algorithm normalization, the data set is first projected into a feature space using at least one transform model. The transform model(s) are locally trained on proprietary datasets to recognize and embed identified features. These models may be subjected to federated training as well. The trained transform models are then deployed at runtime sites. These runtime sites are substantiated in secure computing environments. A runtime dataset is pre-processed by the transform model. Subsequently, the pre-processed/normalized data can be utilized to train a subsequent runtime algorithm or used as input data for an already trained runtime algorithm. When training the runtime algorithm, federated training across each runtime site may be performed.

The features that are identified within the feature space may be those that impact the accuracy of the runtime models. In some cases, the runtime data and training data are images. In these cases the transform model is an image embedded model.

In other embodiments, systems and methods of pre-certifying the runtime mode are presented. This pre-certification is for leakage detection of protected information. The model is received along with a known dataset. The model operates on the dataset to generate an output which is analyzed for exfiltration (either in the output itself or in its weight space). Algorithms that allow for data leakage are flagged. Leakage is determined as a binary determination, risk as epsilon or as a threshold risk. Algorithms that do not exhibit any leakage are locked using a signature process and labeled as certified. Certification may occur in specialized certification environments, which may themselves be secure computing clusters. The locked algorithm may be deployed in runtime environments, which constitute secure computing enclaves. Within these environments, the locked algorithm may be attested and used to process local datasets. In some embodiments a detection model may be trained using the output of models and known datasets. Detection models may undergo federated training. The leakage detection model is deployable in a runtime site where any runtime algorithm may be analyzed by the detection model. Protected data found in the output may be prohibited from release by the detection model. The detection model may be dynamic—the permissions may adjust the kind of data that is determined to be ‘protected’.

In yet another embodiment, systems and methods for processing data using a foundational model for data curation is provided. The foundational model may consume two out of three of the following: a data specification, curated data, and a data set. When the inputs include the data specification and the data set, the system then generates the curated data set using the generative AI model. When the inputs include the data set and the curated data, the system then generates the data specification using the generative AI model. When the inputs include the data specification and the curated data set, the system then generates a validation using the generative AI model.

In yet another embodiment, systems and methods for report generation are provided. In this system the generalized tests error is calculated for the processing of an algorithm on a data set, wherein the processing is performed in a secure computing environment. A dynamic threshold is also calculated, wherein the dynamic threshold is dependent upon the nature of protected information that is present in an output and the intended deployment of the output. The generalized tests error is compared to the dynamic threshold to calculate a report, which is provided to a data steward. This informs the data steward whether or not to proceed with processing a data set of the data steward using the algorithm responsive to the report. Intended deployment is determined by the intended audience. Different data types are provided with different classifications, and the nature of the protected information is responsive to these classifications. The data types include protected health information (PHI), which may be further subdivided by sensitivity levels. The report is either a binary report or a risk gradient report.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIGS. 1A and 1B are example block diagrams of a system for zero trust computing of data by an algorithm, in accordance with some embodiment;

FIG. 2 is an example block diagram showing the core management system, in accordance with some embodiment;

FIG. 3 is an example block diagram showing an example model for the confidential computing data flow, in accordance with some embodiment;

FIG. 4 is a flowchart for an example process for the operation of the confidential computing data processing system, in accordance with some embodiment;

FIG. 5 a flowchart for an example process of acquiring and curating data, in accordance with some embodiment;

FIG. 6 a flowchart for an example process of onboarding a new host data steward, in accordance with some embodiment;

FIG. 7 is a flowchart for an example process of encapsulating the algorithm and data, in accordance with some embodiment;

FIG. 8 is a flowchart for an example process of algorithm encryption and handling, in accordance with some embodiment;

FIG. 9 is an example block diagram showing a trusted computing environment where data normalization is performed before algorithm processing, in accordance with some embodiment;

FIG. 10 is a flowchart for an example process of data normalization and processing, in accordance with some embodiment;

FIG. 11 is an example block diagram showing a certification body for algorithm pre-certification, in accordance with some embodiment;

FIG. 12 is a flowchart for an example process of algorithm pre-certification and usage, in accordance with some embodiment;

FIG. 13 is an example block diagram showing a trusted computing environment with leakage detection model generation, in accordance with some embodiment;

FIG. 14 is a flowchart for an example process of training of a leakage detection model, in accordance with some embodiment;

FIG. 15 is an example block diagram showing a data curator/transformer, in accordance with some embodiment;

FIG. 16 is a flowchart for an example process of data curation and/or data specification or validation generation, in accordance with some embodiment;

FIG. 17 is an example block diagram showing a trusted computing environment with test error calculation for report generation, in accordance with some embodiment;

FIG. 18 is a flowchart for an example process of security report generation and workflow approval, in accordance with some embodiment; and

FIGS. 19A and 19B are illustrations of computer systems capable of implementing the confidential computing, in accordance with some embodiments.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

The present invention relates to systems and methods for the confidential computing application on one or more algorithms processing sensitive datasets. Such systems and methods may be applied to any given dataset, but may have particular utility within the healthcare setting, where the data is extremely sensitive. As such, the following descriptions will center on healthcare use cases. This particular focus, however, should not artificially limit the scope of the invention. For example, the information processed may include sensitive industry information, financial, payroll or other personally identifiable information, or the like. As such, while much of the disclosure will refer to protected health information (PHI) it should be understood that this may actually refer to any sensitive type of data. Likewise, while the data stewards are generally thought to be a hospital or other healthcare entity, these data stewards may in reality be any entity that has and wishes to process their data within a zero-trust environment.

In some embodiments, the following disclosure will focus upon the term “algorithm”. It should be understood that an algorithm may include machine learning (ML) models, neural network models, or other artificial intelligence (AI) models. However, algorithms may also apply to more mundane model types, such as linear models, least mean squares, or any other mathematical functions that convert one or more input values, and results in one or more output models.

Also, in some embodiments of the disclosure, the terms “node”, “infrastructure” and “enclave” may be utilized. These terms are intended to be used interchangeably and indicate a computing architecture that is logically distinct (and often physically isolated). In no way does the utilization of one such term limit the scope of the disclosure, and these terms should be read interchangeably.

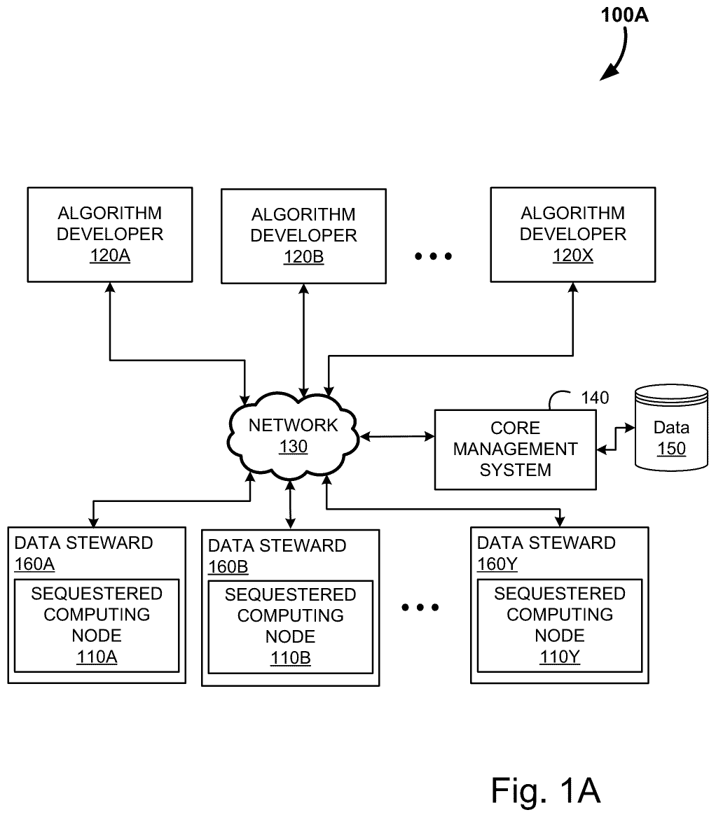

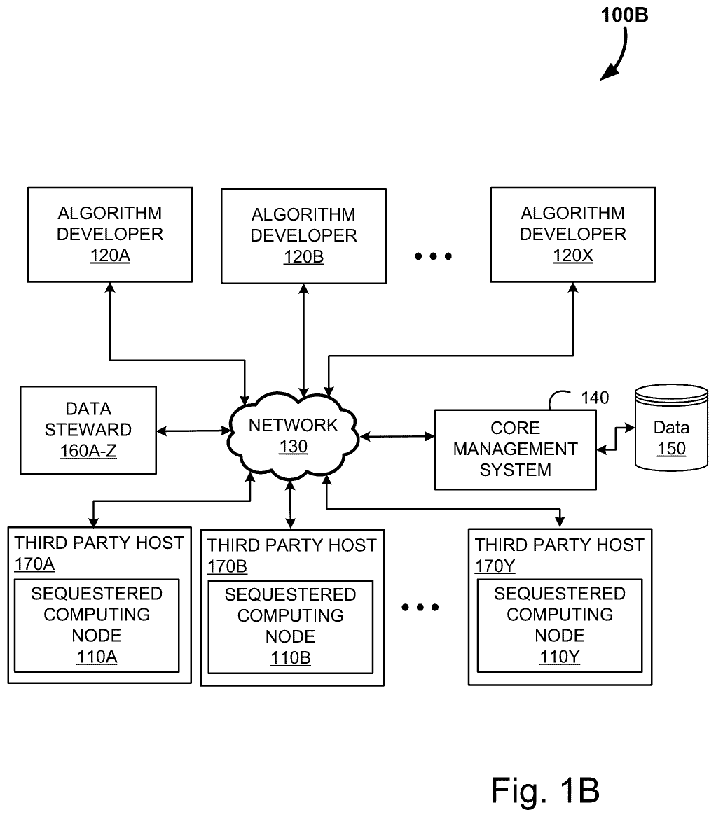

To facilitate discussions, FIG. 1A is an example of a confidential computing infrastructure, shown generally at 100a. This infrastructure includes one or more algorithm developers 120a-x which generate one or more algorithms for processing of data, which in this case is held by one or more data stewards 160a-y. The algorithm developers are generally companies that specialize in data analysis, and are often highly specialized in the types of data that are applicable to their given models/algorithms. However, sometimes the algorithm developers may be individuals, universities, government agencies, or the like. By uncovering powerful insights in vast amounts of information, AI and machine learning (ML) can improve care, increase efficiency, and reduce costs. For example, AI analysis of chest x-rays predicted the progression of critical illness in COVID-19. In another example, an image-based deep learning model developed at MIT can predict breast cancer up to five years in advance. And yet another example is an algorithm developed at University of California San Francisco, which can detect pneumothorax (collapsed lung) from CT scans, helping prioritize and treat patients with this life-threatening condition—the first algorithm embedded in a medical device to achieve FDA approval.

Likewise, the data stewards may include public and private hospitals, companies, universities, banks and other financial institutions, governmental agencies, or the like. Indeed, virtually any entity with access to sensitive data that is to be analyzed may be a data steward.

The generated algorithms are encrypted at the algorithm developer in whole, or in part, before transmitting to the data stewards, in this example ecosystem. The algorithms are transferred via a core management system 140, which may supplement or transform the data using a localized datastore 150. The core management system also handles routing and deployment of the algorithms. The datastore may also be leveraged for key management in some embodiments that will be discussed in greater detail below.

Each of the algorithm developer 120a-x, and the data stewards 160a-y and the core management system 140 may be coupled together by a network 130. In most cases the network is comprised of a cellular network and/or the internet. However, it is envisioned that the network includes any wide area network (WAN) architecture, including private WAN's, or private local area networks (LANs) in conjunction with private or public WANs.

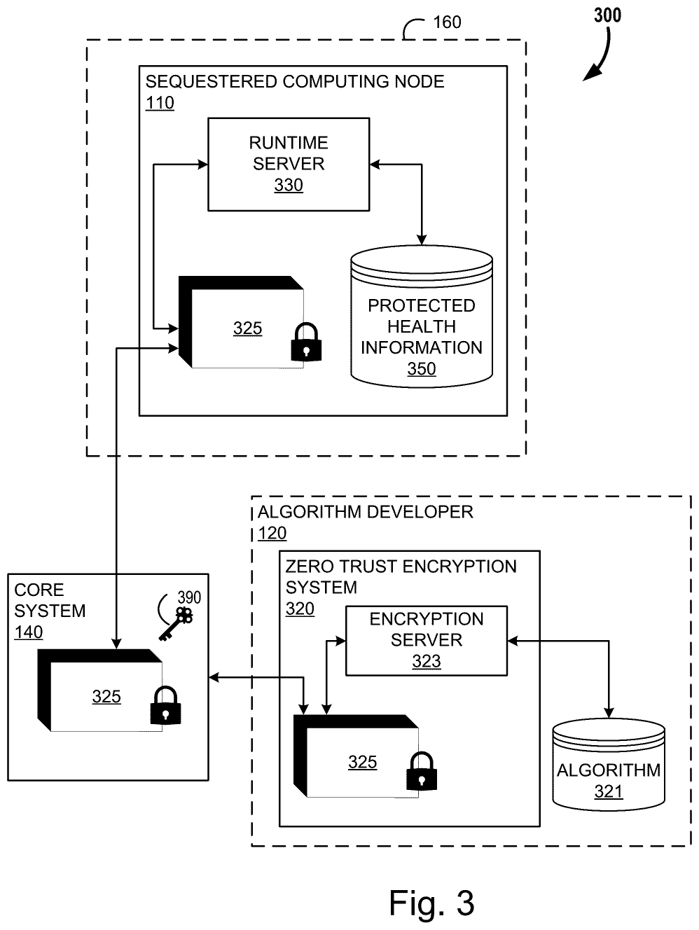

In this particular system, the data stewards maintain sequestered computing nodes 110a-y which function to actually perform the computation of the algorithm on the dataset. The sequestered computing nodes, or “enclaves”, may be physically separate computer server systems, or may encompass virtual machines operating within a greater network of the data steward's systems. The sequestered computing nodes should be thought of as a vault. The encrypted algorithm and encrypted datasets are supplied to the vault, which is then sealed. Encryption keys 390, as seen in FIG. 3, unique to the vault are then provided, which allows the decryption of the data and models to occur. No party has access to the vault at this time, and the algorithm is able to securely operate on the data. The data and algorithms may then be destroyed, or maintained as encrypted, when the vault is “opened” in order to access the report/output derived from the application of the algorithm on the dataset. Due to the specific sequestered computing node being required to decrypt the given algorithm(s) and data, there is no way they can be intercepted and decrypted. This system relies upon public-private key techniques, where the algorithm developer utilizes the public key 390 for encryption of the algorithm, and the sequestered computing node includes the private key in order to perform the decryption. In some embodiments, the private key may be hardware (in the case of Azure, for example) or software linked (in the case of AWS, for example). In other embodiments, the algorithm may be encrypted using a symmetric key, and the symmetric key may be wrapped encrypted by a public key. Specifically, the algorithm developer has their own symmetrical key (content encryption key) used to encrypt the algorithm. The algorithm developer uses the public key to encrypt or “wrap” the content encryption key. The unwrapping occurs in the vault using the private half of the key, to then enable the content encryption key to decrypt the algorithm.

In some particular embodiments, the system sends algorithm models via an Azure Confidential Computing environment to a data steward's environment. Upon verification, the model and the data entered the Intel SGX sequestered enclave where the model is able to be validated against the protected information, for example PHI, data sets. Throughout the process, the algorithm owner cannot see the data, the data steward cannot see the algorithm model, and the management core can see neither the data nor the model. It should be noted that an Intel SGX enclave is but one substantiation of a hardware enabled trusted execution environment. Other hardware and/or software enabled trusted execution environments may be readily employed in other embodiments.

The data steward uploads encrypted data to their cloud environment using an encrypted connection that terminates inside an Intel SGX-sequestered enclave. In some embodiments, the encrypted data may go into Blob storage prior to terminus in the sequestered enclave, where it is pulled upon as needed. Then, the algorithm developer submits an encrypted, containerized AI model which also terminates into an Intel SGX-sequestered enclave. In some specific embodiments, a key management system in the management core enables the containers to authenticate and then run the model on the data within the enclave. In alternate embodiments, where distributed keys are utilized, there is no need for a key management system. Rather in such embodiments, the system is fully distributed among the parties, as shall be described in greater detail below. The data steward never sees the algorithm inside the container and the data is never visible to the algorithm developer. Neither component leaves the enclave. After the model runs, in some embodiments the developer receives a performance report on the values of the algorithm's performance. Finally, the algorithm owner may request that an encrypted artifact containing information about validation results is stored for regulatory compliance purposes and the data and the algorithm are wiped from the system.

FIG. 1B provides a similar ecosystem 100b. This ecosystem also includes one or more algorithm developers 120a-x, which generate, encrypt and output their models. The core management system 140 receives these encrypted payloads, and in some embodiments, transforms or augments unencrypted portions of the payloads. The major difference between this substantiation and the prior figure, is that the sequestered computing node(s) 110a-y are present within a third-party host 170a-y. An example of a third-party host may include an offsite server such as Amazon Web Service (AWS) or similar cloud infrastructure. Other examples can include any network-connected environment, such as traditional data centers. In such situations, the data steward encrypts their dataset(s) and provides them, via the network, to the third party hosted sequestered computing node(s) 110a-y. The output of the algorithm running on the dataset is then transferred from the sequestered computing node in the third-party, back via the network to the data steward (or potentially some other recipient).

In some specific embodiments, the system relies on a unique combination of software and hardware available through Azure Confidential Computing. The solution uses virtual machines (VMs) running on specialized Intel processors with Intel Software Guard Extension (SGX), in this embodiment, running in the third-party system. Intel SGX creates sequestered portions of the hardware's processor and memory known as “enclaves” making it impossible to view data or code inside the enclave. Software within the management core handles encryption, key management, and workflows.

In some embodiments, the system may be some hybrid between FIGS. 1A and 1B. For example, some datasets may be processed at local sequestered computing nodes, especially extremely large datasets, and others may be processed at third parties. Such systems provide flexibility based upon computational infrastructure, while still ensuring all data and algorithms remain sequestered and not visible except to their respective owners.

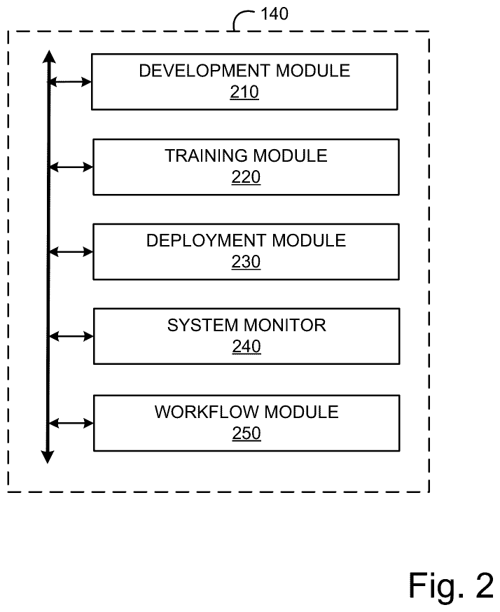

Turning now to FIG. 2, greater detail is provided regarding the core management system 140. The core management system 140 may include a data science development module 210, a data harmonizer workflow creation module 250, a software deployment module 230, a federated master algorithm training module 220, a system monitoring module 240, and a data store comprising global join data 240.

The data science development module 210 may be configured to receive input data requirements from the one or more algorithm developers for the optimization and/or validation of the one or more models. The input data requirements define the objective for data curation, data transformation, and data harmonization workflows. The input data requirements also provide constraints for identifying data assets acceptable for use with the one or more models. The data harmonizer workflow creation module 250 may be configured to manage transformation, harmonization, and annotation protocol development and deployment. The software deployment module 230 may be configured along with the data science development module 210 and the data harmonizer workflow creation module 250 to assess data assets for use with one or more models. This process can be automated or can be an interactive search/query process. The software deployment module 230 may be further configured along with the data science development module 210 to integrate the models into a sequestered capsule computing framework, along with required libraries and resources.

In some embodiments, it is desired to develop a robust, superior algorithm/model that has learned from multiple disjoint private data sets (e.g., clinical and health data) collected by data hosts from sources (e.g., patients). The federated master algorithm training module may be configured to aggregate the learning from the disjoint data sets into a single master algorithm. In different embodiments, the algorithmic methodology for the federated training may be different. For example, sharing of model parameters, ensemble learning, parent-teacher learning on shared data and many other methods may be developed to allow for federated training. The privacy and security requirements, along with commercial considerations such as the determination of how much each data system might be paid for access to data, may determine which federated training methodology is used.

The system monitoring module 240 monitors activity in sequestered computing nodes. Monitored activity can range from operational tracking such as computing workload, error state, and connection status as examples to data science monitoring such as amount of data processed, algorithm convergence status, variations in data characteristics, data errors, algorithm/model performance metrics, and a host of additional metrics, as required by each use case and embodiment.

In some instances, it is desirable to augment private data sets with additional data located at the core management system (join data 150). For example, geolocation air quality data could be joined with geolocation data of patients to ascertain environmental exposures. In certain instances, join data may be transmitted to sequestered computing nodes to be joined with their proprietary datasets during data harmonization or computation.

The sequestered computing nodes may include a harmonizer workflow module 250, harmonized data, a runtime server, a system monitoring module, and a data management module (not shown). The transformation, harmonization, and annotation workflows managed by the data harmonizer workflow creation module may be deployed by and performed in the environment by harmonizer workflow module using transformations and harmonized data. In some instances, the join data may be transmitted to the harmonizer workflow module to be joined with data during data harmonization. The runtime server may be configured to run the private data sets through the algorithm/model.

The system monitoring module monitors activity in the sequestered computing node. Monitored activity may include operational tracking such as algorithm/model intake, workflow configuration, and data host onboarding, as required by each use case and embodiment. The data management module may be configured to import data assets such as private data sets while maintaining the data assets within the pre-exiting infrastructure of the data stewards.

Turning now to FIG. 3, an example of the flow of algorithms and data are provided, generally at 300. The Zero-Trust Encryption System 320 manages the encryption, by an encryption server 323, of all the algorithm developer's 120 software assets 321 in such a way as to prevent exposure of intellectual property (including source or object code) to any outside party, including the entity running the core management system 140 and any affiliates, during storage, transmission and runtime of said encrypted algorithms 325. In this embodiment, the algorithm developer is responsible for encrypting the entire payload 325 of the software using its own encryption keys. Decryption is only ever allowed at runtime in a sequestered capsule computing environment 110.

The core management system 140 receives the encrypted computing assets (algorithms) 325 from the algorithm developer 120. Decryption keys to these assets are not made available to the core management system 140 so that sensitive materials are never visible to it. The core management system 140 distributes these assets 325 to a multitude of data steward nodes 160 where they can be processed further, in combination with private datasets, such as protected health information (PHI) 350.

Each Data Steward Node 160 maintains a sequestered computing node 110 that is responsible for allowing the algorithm developer's encrypted software assets 325 (the “algorithm” or “algo”) to compute on a local private dataset 350 that is initially encrypted. Within data steward node 160, one or more local private datasets (not illustrated) is harmonized, transformed, and/or annotated and then this dataset is encrypted by the data steward, into a local dataset 350, for use inside the sequestered computing node 110.

The sequestered computing node 110 receives the encrypted software assets 325 and encrypted data steward dataset(s) 350 and manages their decryption in a way that prevents visibility to any data or code at runtime at the runtime server 330. In different embodiments this can be performed using a variety of secure computing enclave technologies, including but not limited to hardware-based and software-based isolation.

In this present embodiment, the entire algorithm developer software asset payload 325 is encrypted in a way that it can only be decrypted in an approved sequestered computing enclave/node 110. This approach works for sequestered enclave technologies that do not require modification of source code or runtime environments in order to secure the computing space (e.g., software-based secure computing enclaves).

The Algorithm developer 120 generates an algorithm, which is then encrypted and provided as an encrypted algorithm payload 325 to the core management system 140. As discussed previously, the core management system 140 is incapable of decrypting the encrypted algorithm 325. Rather, the core management system 140 controls the routing of the encrypted algorithm 325 and the management of keys. The encrypted algorithm 325 is then provided to the data steward 160 which is then “placed” in the sequestered computing node 110. The data steward 160 is likewise unable to decrypt the encrypted algorithm 325 unless and until it is located within the sequestered computing node 110, in which case the data steward still lacks the ability to access the “inside” of the sequestered computing node 110. As such, the algorithm is never accessible to any entity outside of the algorithm developer.

Likewise, the data steward 160 has access to protected health information and/or other sensitive information. The data steward 160 never is required to transfer this data outside of its ecosystem (an if it is, it may remain in an encrypted state) thus ensuring that the data is always inaccessible by any other party by virtue of it remaining encrypted when accessible by any other party. The sensitive data may be encrypted (or remain in the clear) as it is also transferred into the sequestered computing node 110. This data store is made accessible to the runtime server 330 also located “inside” the sequestered computing node 110. The runtime server 330 decrypts the encrypted algorithm 325 to yield the underlying algorithm model. This algorithm may then use the data store to generate inferences regarding the date contained in the data store (not illustrated). These inferences have value for the data steward 110 as well as other interested parties and may be outputted to the data steward (or other interested parties such as researchers or regulators) for consumption. The runtime server 330 may likewise engage in training activities.

The runtime server 330 may also perform a number of other operations, such as the generation of a performance model or the like. The performance model is a regression model generated based upon the inferences derived from the algorithm. The performance model provides data regarding the performance of the algorithm based upon the various inputs. The performance model may model for any of algorithm accuracy, F1 score, precision, recall, dice score, ROC (receiver operator characteristic) curve/area, log loss, Jaccard index, error, R2, by some combination thereof, or by any other suitable metric.

Once the algorithm developer 120 receives the performance model it may be decrypted, and leveraged to validate the algorithm and, importantly, may be leveraged to actively train the algorithm in the future. This may occur by identifying regions of the performance model that have lower performance ratings and identify attributes/variables in the datasets that correspond to these poorer performing model segments. The system then incorporates human feedback when such variables are present in a dataset to assist in generating a gold standard training set for these variable combinations. The performance model may then be trained based upon these gold standard training sets. Even without the generation of additional gold standard data, investigation of poorer performing model segments enables changes to the functional form of the model and testing for better performance. It is likewise possible that the inclusion of additional variables by the model allows for the distinction of attributes of a patient population. This is identified by areas of the model that has a lower performance which indicates that there is a fundamental issue with the model. An example is that a model operates well (has higher performance) for male patients as compared to female patients. This may indicate that different model mechanics may be required for female patient populations.

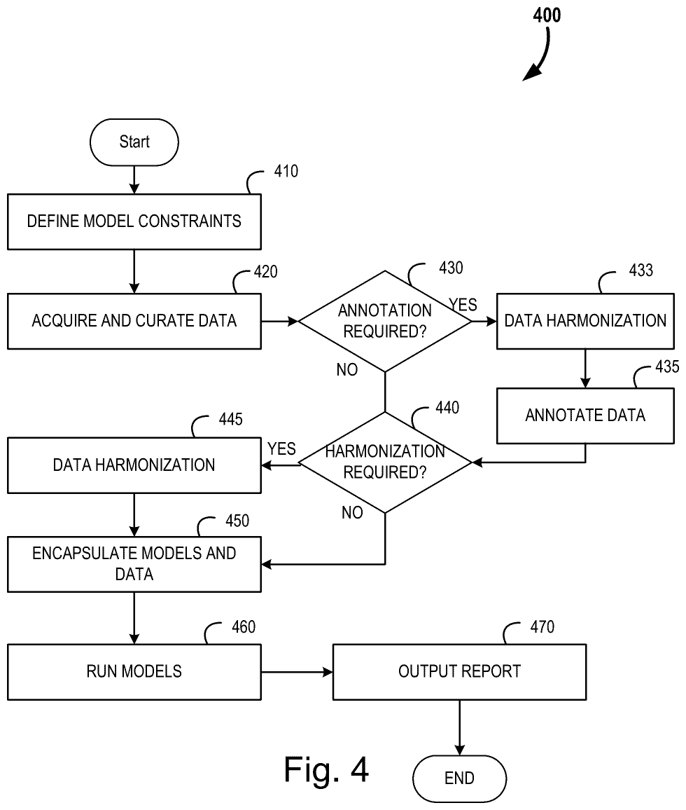

Turning to FIG. 4, one embodiment of the process for deployment and running of algorithms within the sequestered computing nodes is illustrated, at 400. Initially the algorithm developer provides the algorithm to the system using wheatever process they locally employ. For example, at least one algorithm/model is generated by the algorithm developer using their own development environment, tools, and seed data sets (e.g., training/testing data sets). In some embodiments, the algorithms may be trained on external datasets instead, as will be discussed further below. The algorithm developer provides constraints (at 410) for the optimization and/or validation of the algorithm(s). Constraints may include any of the following: (i) training constraints, (ii) data preparation constraints, and (iii) validation constraints. These constraints define objectives for the optimization and/or validation of the algorithm(s) including data preparation (e.g., data curation, data transformation, data harmonization, and data annotation), model training, model validation, and reporting.

In some embodiments, the training constraints may include, but are not limited to, at least one of the following: hyperparameters, regularization criteria, convergence criteria, algorithm termination criteria, training/validation/test data splits defined for use in algorithm(s), and training/testing report requirements. A model hyper parameter is a configuration that is external to the model, and which value cannot be estimated from data. The hyperparameters are settings that may be tuned or optimized to control the behavior of a ML or AI algorithm and help estimate or learn model parameters.

Regularization constrains the coefficient estimates towards zero. This discourages the learning of a more complex model in order to avoid the risk of overfitting. Regularization, significantly reduces the variance of the model, without a substantial increase in its bias. The convergence criterion is used to verify the convergence of a sequence (e.g., the convergence of one or more weights after a number of iterations). The algorithm termination criteria define parameters to determine whether a model has achieved sufficient training. Because algorithm training is an iterative optimization process, the training algorithm may perform the following steps multiple times. In general, termination criteria may include performance objectives for the algorithm, typically defined as a minimum amount of performance improvement per iteration or set of iterations.

The training/testing report may include criteria that the algorithm developer has an interest in observing from the training, optimization, and/or testing of the one or more models. In some instances, the constraints for the metrics and criteria are selected to illustrate the performance of the models. For example, the metrics and criteria such as mean percentage error may provide information on bias, variance, and other errors that may occur when finalizing a model such as vanishing or exploding gradients. Bias is an error in the learning algorithm. When there is high bias, the learning algorithm is unable to learn relevant details in the data. Variance is an error in the learning algorithm, when the learning algorithm tries to over-learn from the dataset or tries to fit the training data as closely as possible. Further, common error metrics such as mean percentage error and R2 score are not always indicative of accuracy of a model, and thus the algorithm developer may want to define additional metrics and criteria for a more in depth look at accuracy of the model.

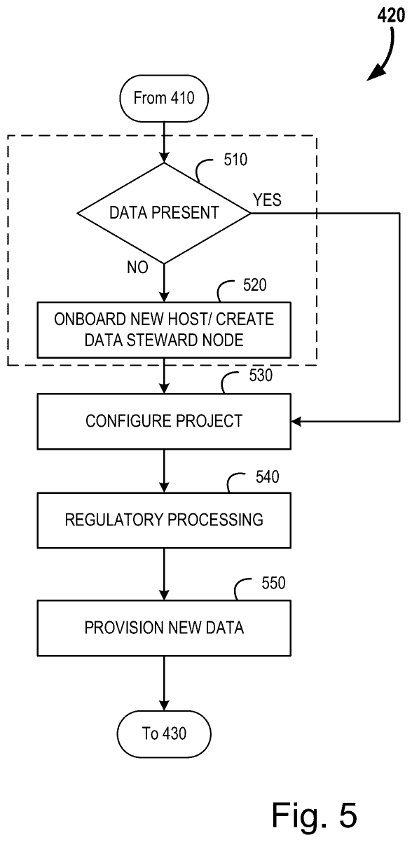

Next, data assets that will be subjected to the algorithm(s) are identified, acquired, and curated (at 420). FIG. 5 provides greater detail of this acquisition and curation of the data. Often, the data may include healthcare related data (PHI). Initially, there is a query if data is present (at 510). The identification process may be performed automatically by the platform running the queries for data assets (e.g., running queries on the provisioned data stores using the data indices) using the input data requirements as the search terms and/or filters. Alternatively, this process may be performed using an interactive process, for example, the algorithm developer may provide search terms and/or filters to the platform. The platform may formulate questions to obtain additional information, the algorithm developer may provide the additional information, and the platform may run queries for the data assets (e.g., running queries on databases of the one or more data hosts or web crawling to identify data hosts that may have data assets) using the search terms, filters, and/or additional information. In either instance, the identifying is performed using differential privacy for sharing information within the data assets by describing patterns of groups within the data assets while withholding private information about individuals in the data assets.

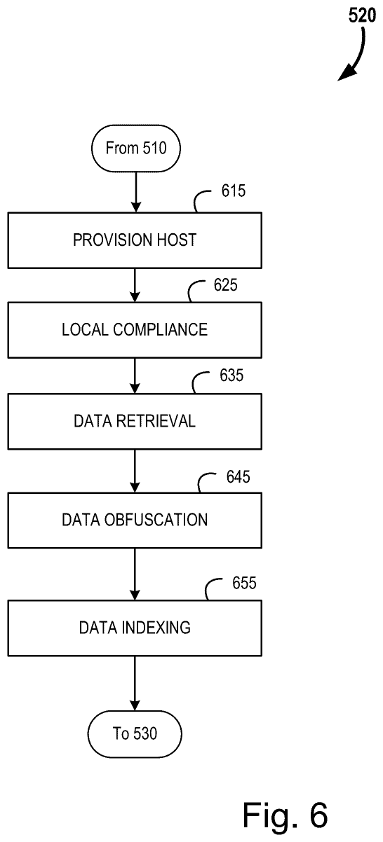

If the assets are not available, the process generates a new data steward node (at 520). The data query and onboarding activity (surrounded by a dotted line) is illustrated in this process flow of acquiring the data; however, it should be realized that these steps may be performed any time prior to model and data encapsulation (step 450 in FIG. 6). Onboarding/creation of a new data steward node is shown in greater detail in relation to FIG. 6. In this example process a data host compute and storage infrastructure (e.g., a sequestered computing node as described with respect to FIGS. 1A-5) is provisioned (at 615) within the infrastructure of the data steward. In some instances, the provisioning includes deployment of encapsulated algorithms in the infrastructure, deployment of a physical computing device with appropriately provisioned hardware and software in the infrastructure, deployment of storage (physical data stores or cloud-based storage), or deployment on public or private cloud infrastructure accessible via the infrastructure, etc.

Next, governance and compliance requirements are performed (at 625). In some instances, the governance and compliance requirements includes getting clearance from an institutional review board, and/or review and approval of compliance of any project being performed by the platform and/or the platform itself under governing law such as the Health Insurance Portability and Accountability Act (HIPAA). Subsequently, the data assets that the data steward desires to be made available for optimization and/or validation of algorithm(s) are retrieved (at 635). In some instances, the data assets may be transferred from existing storage locations and formats to provisioned storage (physical data stores or cloud-based storage) for use by the sequestered computing node (curated into one or more data stores). The data assets may then be obfuscated (at 645). Data obfuscation is a process that includes data encryption or tokenization, as discussed in much greater detail below. Lastly, the data assets may be indexed (at 655). Data indexing allows queries to retrieve data from a database in an efficient manner. The indexes may be related to specific tables and may be comprised of one or more keys or values to be looked up in the index (e.g., the keys may be based on a data table's columns or rows).

Returning to FIG. 5, after the creation of the new data steward, the project may be configured (at 530). In some instances, the data steward computer and storage infrastructure is configured to handle a new project with the identified data assets. In some instances, the configuration is performed similarly to the process described of FIG. 6. Next, regulatory approvals (e.g., IRB and other data governance processes) are completed and documented (at 540). Lastly, the new data is provisioned (at 550). In some instances, the data storage provisioning includes identification and provisioning of a new logical data storage location, along with creation of an appropriate data storage and query structure.

Returning now to FIG. 4, after the data is acquired and configured, a query is performed if there is a need for data annotation (at 430). If so, the data is initially harmonized (at 433) and then annotated (at 435). Data harmonization is the process of collecting data sets of differing file formats, naming conventions, and columns, and transforming it into a cohesive data set. The annotation is performed by the data steward in the sequestered computing node. A key principle to the transformation and annotation processes is that the platform facilitates a variety of processes to apply and refine data cleaning and transformation algorithms, while preserving the privacy of the data assets, all without requiring data to be moved outside of the technical purview of the data steward.

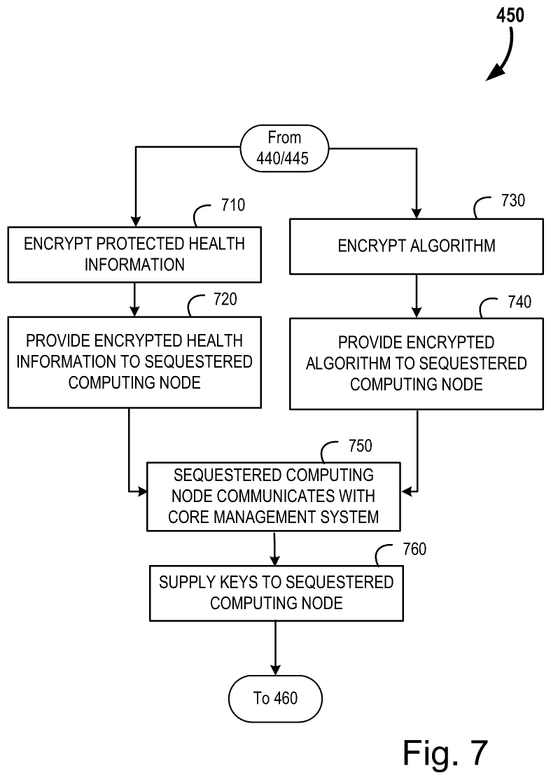

After annotation, or if annotation was not required, another query determines if additional data harmonization is needed (at 440). If so, then there is another harmonization step (at 445) that occurs in a manner similar to that disclosed above. After harmonization, or if harmonization isn't needed, the models and data are encapsulated (at 450). Data and model encapsulation is described in greater detail in relation to FIG. 7. In the encapsulation process the protected data, and the algorithm are each encrypted (at 710 and 730 respectively). In some embodiments, the data is encrypted either using traditional encryption algorithms (e.g., RSA) or homomorphic encryption.

Next the encrypted data and encrypted algorithm are provided to the sequestered computing node (at 720 and 740 respectively). There processes of encryption and providing the encrypted payloads to the sequestered computing nodes may be performed asynchronously, or in parallel. Subsequently, the sequestered computing node may phone home to the core management node (at 750) requesting the keys needed. These keys are then also supplied to the sequestered computing node (at 760), thereby allowing the decryption of the assets.

Returning again to FIG. 4, once the assets are all within the sequestered computing node, they may be decrypted and the algorithm may run against the dataset (at 460). The results from such runtime may be outputted as a report (at 470) for downstream consumption.

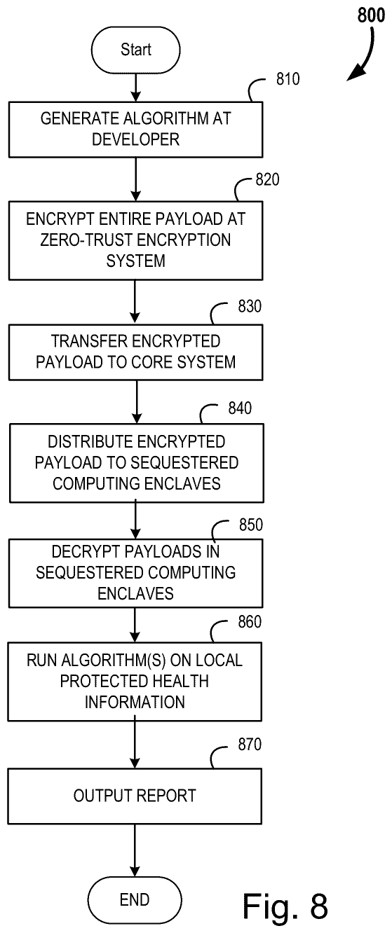

Turning now to FIG. 8, a first embodiment of the system for confidential computing processing of the data assets by the algorithm is provided, at 800. In this example process, the algorithm is initially generated by the algorithm developer (at 810) in a manner similar to that described previously. The entire algorithm, including its container, is then encrypted (at 820), using a public key, by the encryption server within the algorithm developer's infrastructure. The entire encrypted payload is provided to the core management system (at 830). The core management system then distributes the encrypted payload to the sequestered computing enclaves (at 840).

Likewise, the data steward collects the data assets desired for processing by the algorithm. This data is also provided to the sequestered computing node. In some embodiments, this data may also be encrypted. The sequestered computing node then contacts the core management system for the keys. The system relies upon public-private key methodologies for the decryption of the algorithm, and possibly the data (at 850).

After decryption within the sequestered computing node, the algorithm(s) are run (at 860) against the protected health information (or other sensitive information based upon the given use case). The results are then output (at 870) to the appropriate downstream audience (generally the data steward or algorithm developer, but may include public health agencies or other interested parties).

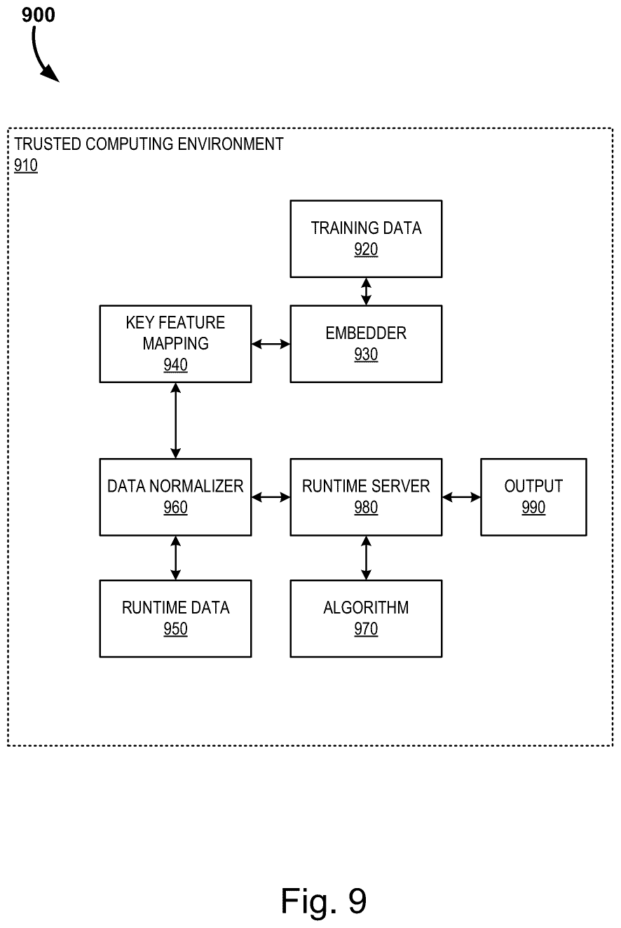

Turning now to FIG. 9, a block diagram of a data normalization system 900 is provided. The trusted computing environment 910 is illustrated as including the training data 920 which is used to model the normalization. This training data may be provided to an embedder 930 which maps the features of the training data set 910 in an abstract vector space. In some embodiments, the embedder may include an image embedding function when the training data includes image data (such as x-ray images or the like). In some embodiments the embedder 930 may constitute a generative AI model. In alternate embodiments, the embedder includes a processing pipeline including a series of transforms to generate a final image, and then a series of additional operations including at least one machine learning model to extract out the feature set.

The embedder generates a series of key features that provide a mapping 940. A data normalizer 960 consumes the key feature mapping 940 and the runtime data 950 that will be processed, and harmonizes the runtime data 950 to generate a normalized data set. This normalization process is a transformation and/or extraction of pertinent data for the algorithm's operation, while eliminating superfluous information and standardizing format of the runtime data. All incoming data sets are filtered and transformed when received to render the feature vector set regardless of source or format, thereby allowing data sets from multiple sources to be analyzed in a consistent manner.

By way of example, suppose the runtime data is x-ray images where a lead marker is placed in the frame to indicate the side and/or body part being imaged. Also assume that the x-ray images have a certain contrast level, and are sized a width of A and a height of B. Other data set may be different (or even different images in the same dataset). For example, other data images may omit the lead marker, or may have an aspect ratio of C width and D height. Contrast, and a myriad of other features may be different between the various sets of data. The normalization step is used to prune out the unimportant/junk information (such as the lead marker) and harmonize other features such as aspect ratio, contrast, resolution, gamma and the like. Similarly, when the data is rather a set of vectors (a series of measurements and other data, for example), the system is trained to identify the vector space that is not impacting the algorithm function, and remove or deemphasize that data. For example, name may be a field in the vector. The person's name has no bearing on the algorithm findings, and can be filtered from the dataset.

Data normalization allows the system to pre-process runtime data and generate a “clean” set of data for the algorithm's usage. The algorithm 970 is provided to the runtime server 980 along with this normalized data. The runtime server securely processes the normalized data through the algorithm, resulting in an output 990 which is more accurate than if the algorithm were operating on raw/dirty data. In addition to accuracy improvements, the usage or normalized data reduces computation complexity and may result in a computer system operating more efficiently by conserving the required processing power. Further, overfitting of the algorithm during training may be reduced by the usage of the normalized data over raw data.

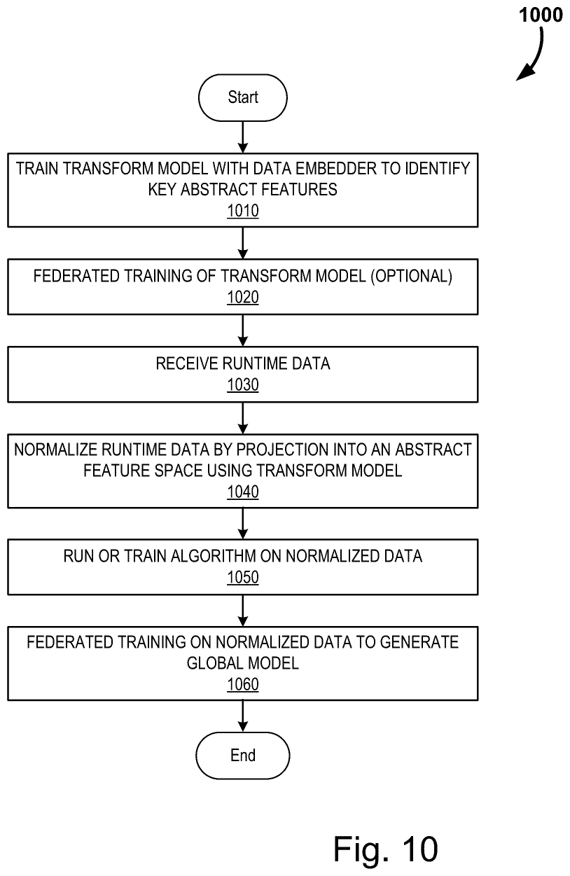

FIG. 10 provides a flow diagram of the example process of data normalization and processing, shown generally at 1000. As noted before, the transform model is trained in an embedder to identify key abstract features (at 1010). Training uses a curated dataset as a prototypical dataset/format. Any raw data sets received will be processed by the transforms and other operations to mimic the curated data set and subsequently extract out this data as a feature vector. Generative AI models may be well situated to assist in this process. Optionally, it is possible to perform federated training of these transform models (at 1020) to improve overall accuracy of the normalizations. The runtime data is received (at 1030) and normalized (at 1040). Data normalization is performed by projecting the runtime data into an abstract feature space using the transform model to generate the feature vector set. This mapping can occur in a standardized manner across all data steward sites regardless of differences in the underlying data.

After normalization, the resulting harmonized data may be used for running an already trained algorithm or may be leveraged to train an untrained algorithm (at 1050). When training is being performed, the trained models may be aggregated to perform federated training and thereby generate a more accurate global model (at 1060).

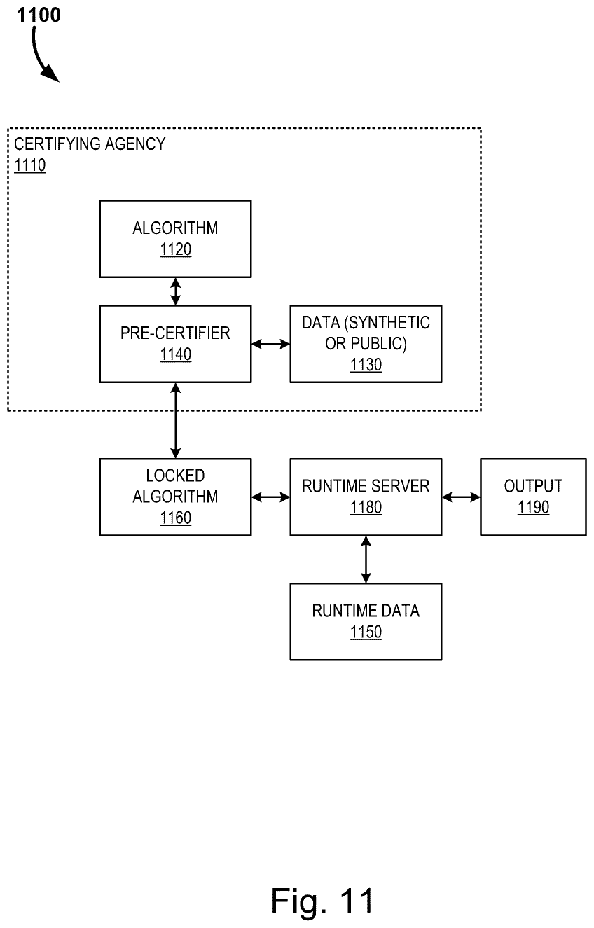

Moving on to FIG. 11, an example block diagram 1100 of a system for algorithm certification is provided. The algorithm 1120 is first consumed by a third party, trusted certifying agency 1110. This is a trained algorithm or raw algorithm. The third party certifying agency may be the core management system, or some other regulator or licensed group. A known data set 1130 is also available to the certifying agency. This known data may include synthetic data, or data that has already become publicly available. A pre-certifier 1140 server consumes the data 1130 and the algorithm 1120. The algorithm (when untrained) may be trained upon at least a subset of the data. The resulting model weights are then compared to the data used to train the model, and any instances of data exfiltration may be flagged. Similarly, trained algorithms may be processed with the data (or a subset of said data) to generate an output report. This report may likewise be analyzed for data exfiltration by the pre-certifier.

Data exfiltration may be overt (often the case when exfiltration is unintentional) or can be more subtle. Overt data exfiltration generally has a model weight and/or output report field showing some portion of the data in the clear. In some cases, when the data being exposed is not protected, and/or is obfuscated such that the owner of the data is indiscernible, this may not be an issue. However, is the exposed data is protected information, the algorithm may be flagged as exfiltrating sensitive data. Subtle data exfiltration, such as encoding data in a weight space of a model, is more complex and harder to detect. More sophisticated reverse models may be employed to take the algorithm weights sets and determine if data can be extracted from them. For example, statistical means whereby model weights may be reconstructed, and/or using prompting of the model to extract private data may be employed. Likewise, membership inference attacks or parameter attacks may be employed. These statistic-based attacks provide a certainty level of a discovery being a member of the training set. The system may be optimized to reduce the level of certainty of a membership of known/real data below a threshold, thus indicating that the inference attack or parameter attack is uncertain of what data underlies the model's training set.

When the algorithm is found to be exfiltrating data that is sensitive, the algorithm may be flagged and rejected. However, if the algorithm does not exfiltrate, the certifying agency 1110 may perform a signature process on the algorithm to generate a locked algorithm 1160.

This locked algorithm 1160 may be consumed by any number of downstream runtime servers 1180 in data stewards secure computing nodes. The runtime data 1150 may be processed using the locked algorithm 1160 to generate useful outputs 1190.

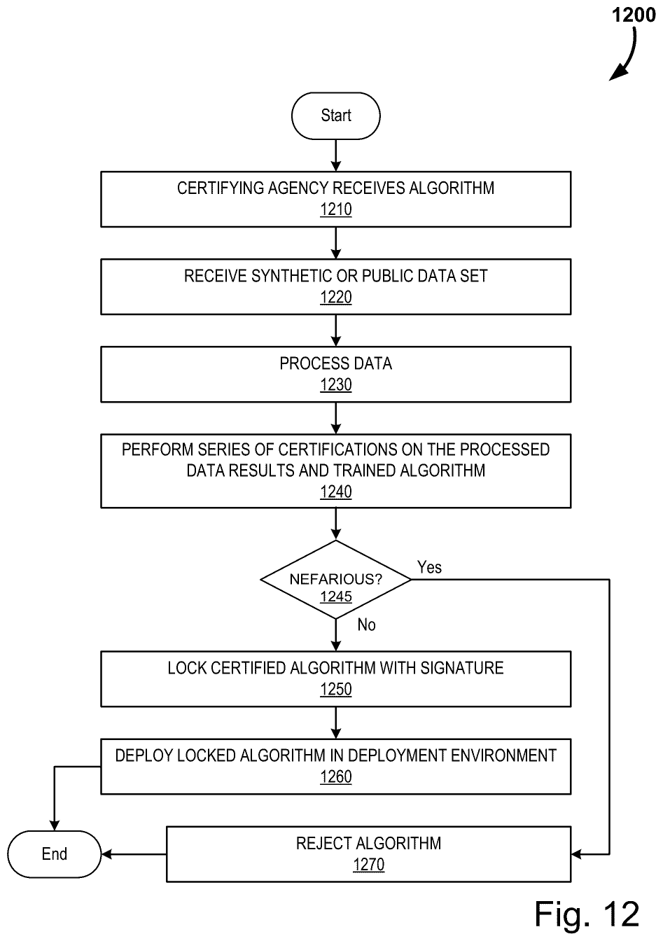

FIG. 12 provides a more detailed flow diagram of an example process of this algorithm certification process, shown generally at 1200. Initially the certifying agency receives an algorithm (at 1210). This algorithm may be an untrained algorithm, or a trained algorithm. The system also receives a data set that is either public or synthetic (at 1220). This data is used with the received algorithm to process the data (at 1230). This processing may include training an untrained algorithm, and/or running a trained algorithm on the data. When the processing is a training step, the resulting weights are the output. Otherwise, when processing the data on the trained algorithm, the resulting output is a report. The system may perform a series of certification tests on the processed data results (report and/or weights) and the trained algorithm (at 1240) in order to certify the algorithm. For example, a series of different tests may be employed to validate that the algorithm is viable (performs at a certain level) and/or resistant to data exfiltration during training.

The system looks for incidences of data exfiltration (intentional or otherwise) through these certification steps and makes a determination if the algorithm is nefarious or not (at 1245). A nefarious algorithm exfiltrates sensitive data, while a benign algorithm does not release any information and/or the information is suitably obfuscated as to be in compliance with privacy regulations. A nefarious algorithm is rejected by the system (at 1270). A benign algorithm is locked via a signature process discussed above (at 1250) and then deployed for downstream processing (at 1260).

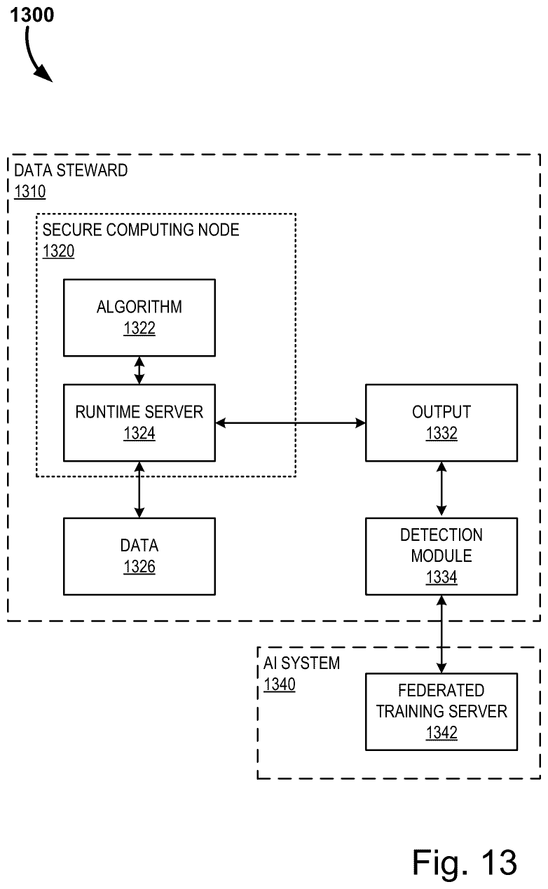

Turning now to FIG. 13, an example block diagram 1300 of a leakage detection system is provided. This leakage detection may employ exfiltration models used in relation to the certification process discussed in FIGS. 11 and 12. However, as opposed to occurring in a certification agency, the present leakage detection process may take place in the data steward 1310. The data steward 1310 includes a secure computing node 1320 within which the algorithm 1322 may operate on a proprietary data set 1326 within a runtime server 1324. The data set is known to the data steward 1310 and can be employed by a detection module 1334 to determine if and when a leakage event occurs. The output 1332 of the runtime server 1324 is fed to the leakage detection model 1334. As the detection module 1334 has access to the data 1326, instances of the data in the output can be readily identified and flagged. The detection model 1334 may be supplied to a federated training server 1342 located in an external AI system 1340. In some embodiments, the AI system 1340 is the core management system. Here the leakage detection models may be aggregated to form a global detection model. This global detection model may be deployed to other data stewards to validate that any given algorithm doesn't exfiltrate data when operating on private data sets.

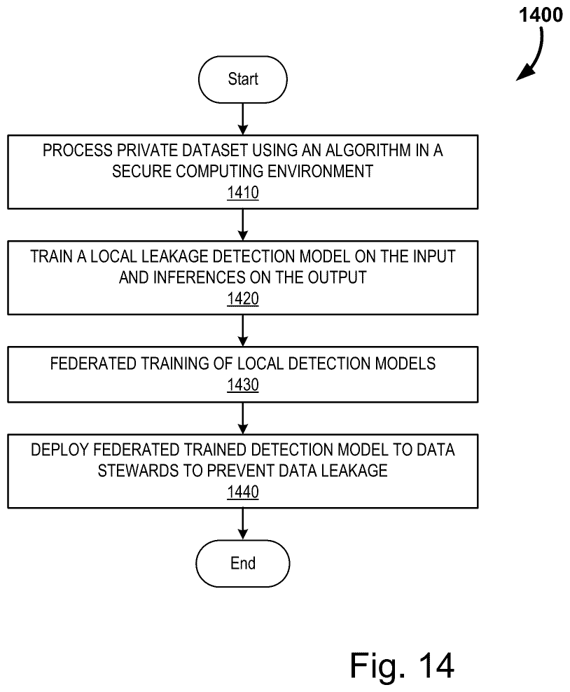

FIG. 14 provides a more detailed flow diagram for the process of leakage detection, shown generally at 1400. This process begins by processing a private dataset using an algorithm within a secure computing environment (at 1410). A local leakage detection model may be trained on the dataset and the inferences contained in the algorithm output (at 1420). The local leakage detection models may be aggregated and used to perform federated training on the various locally trained leakage detection models (at 1430) to generate a global leakage detection model. The global leakage detection model may then be deployed to various data stewards to prevent data leakage from future algorithms (at 1440).

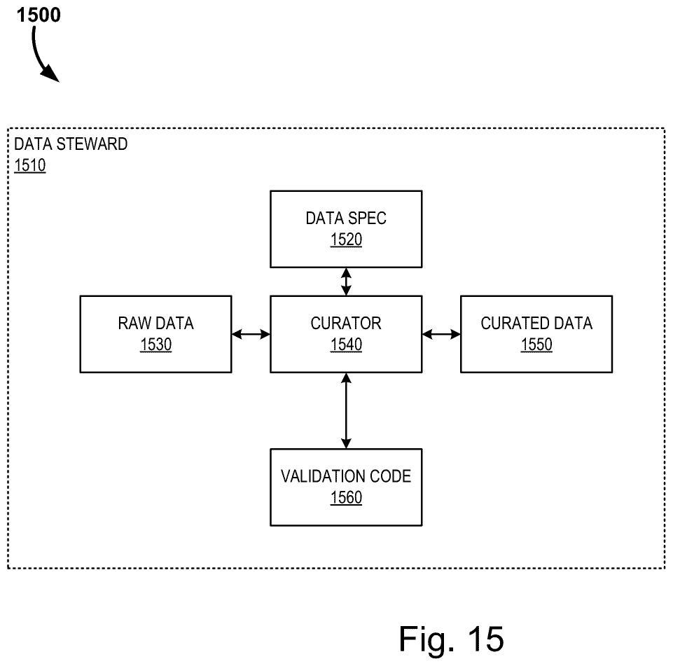

Turning now to FIG. 15, an example block diagram 1500 for data curation is provided. Curated data is important because the algorithm has been designed to operate on a specific data set. Data curation may occur within a data steward 1510. Central to this system is a curator server 1540, which is trained using the raw data and any of the data specification 1520 or curated data 1550 to generate the other. For example, with the data specification 1520, the curator may apply the specification to the raw data 1530 to generate a curated data set 1550. Conversely, with the raw data 1530 and the curated data 1550, the data specification 1520 may be inferred. The curator 1540 itself may employ a foundational model, and in some instances a large language model (LLM), or other generative AI model, in order to perform the curations.

When the data specification 1520 and the curated data 1550 are both available to the curator 1540, the system may be able to generate a validation code 1560 indicating if the curated data 1550 meets the specification requirements.

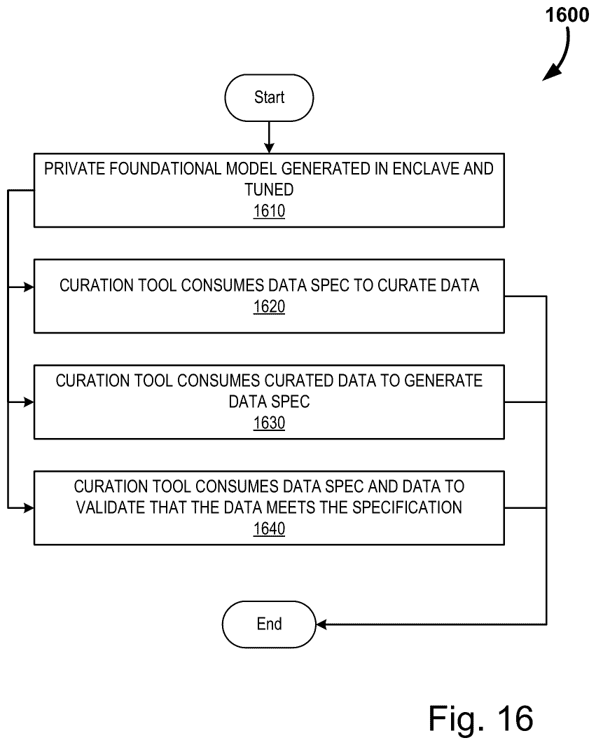

FIG. 16 provides a flow diagram for the example process of data curation, shown generally at 1600. In this process, initially a private foundational model is generated in the enclave and tuned (at 1610). As indicated previously, the curation model may be a LLM or similar foundational model. This model may then be utilized to perform one or more of the following: 1) consume the data specification and raw data to generate a curated data set (at 1620), 2) consume the curated data and raw data to generate the data specification (at 1630), and/or 3) consume the data specification and the curated data to determine if the curated data meets the specification (at 1640) and generating a validation code accordingly.

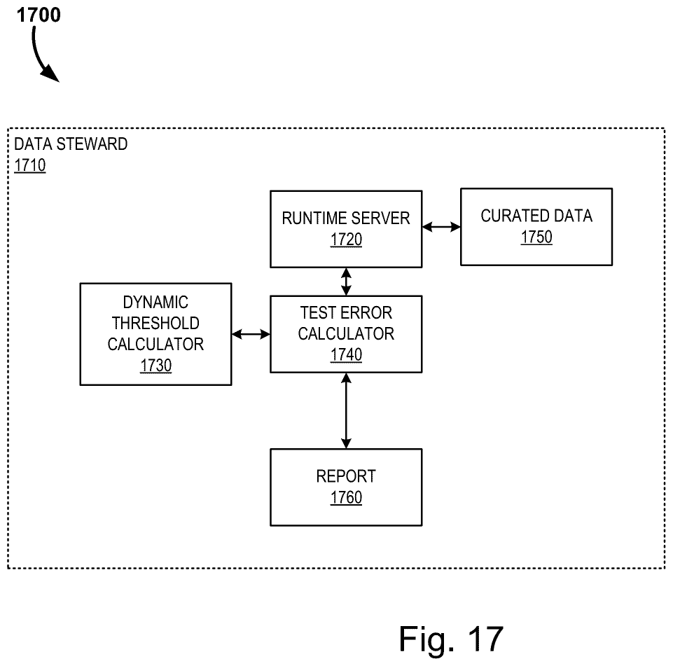

Turning to FIG. 17, an example block diagram 1700 is provided for generating a report on data security. This process occurs within the data steward 1710, where the runtime server 1720 receives curated data 1750 and processes it to generate an output. The output is then consumed by a test error calculator 1740 which determines the test error of the output. Overfitting a model is related to memorization by the model and lack of generalization of the model. The gap between the training performance and the test performance defines the degree of overfitting and is the test error calculation. Low gap between the training performance and test performance indicates a large degree of generalization and a low likelihood of memorization. However, as the difference increases, the likelihood of data memorization increases.

A dynamic threshold calculator 1730 sets a base threshold for the test error. The threshold may be data set specific, use case specific, and/or the model type (e.g., clustering model versus mixture model with a high degree of overlap). The calculated test error is then compared against the threshold and a report 1760 is generated. This report may be a binary determination of whether the algorithm is “secure” or not, or may be a gradient indicating the degree of ability to attack the model.

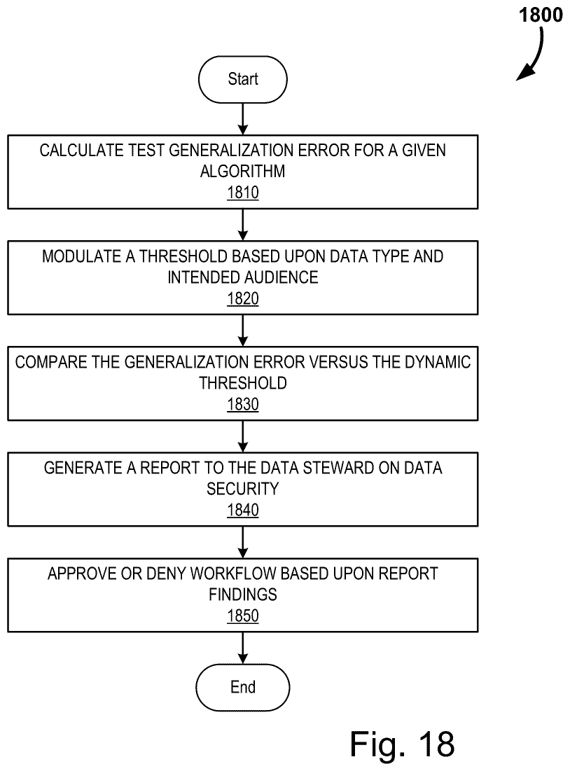

FIG. 18 provides a flow diagram for the example process of the report generation, shown generally at 1800. In this example process, the test generalization error for a given algorithm is first calculated (at 1810). A threshold is modulated based upon the data type and intended audience (at 1820). For example, for a regulator, the threshold may be set higher than for consumption by the general public. Likewise, more sensitive information, such as names and other personally identifiable information may be set at a higher threshold than basic de-identified vital information (such as heartrate).

The generalization error is compared against this dynamic threshold (at 1830). A report is generated on the level of data security based on this comparison (at 1840). The report is provided to the data steward, thus informing additional workflow decision processes (at 1850). For example, if the security level is too low, the system may prevent a downstream workflow from occurring.

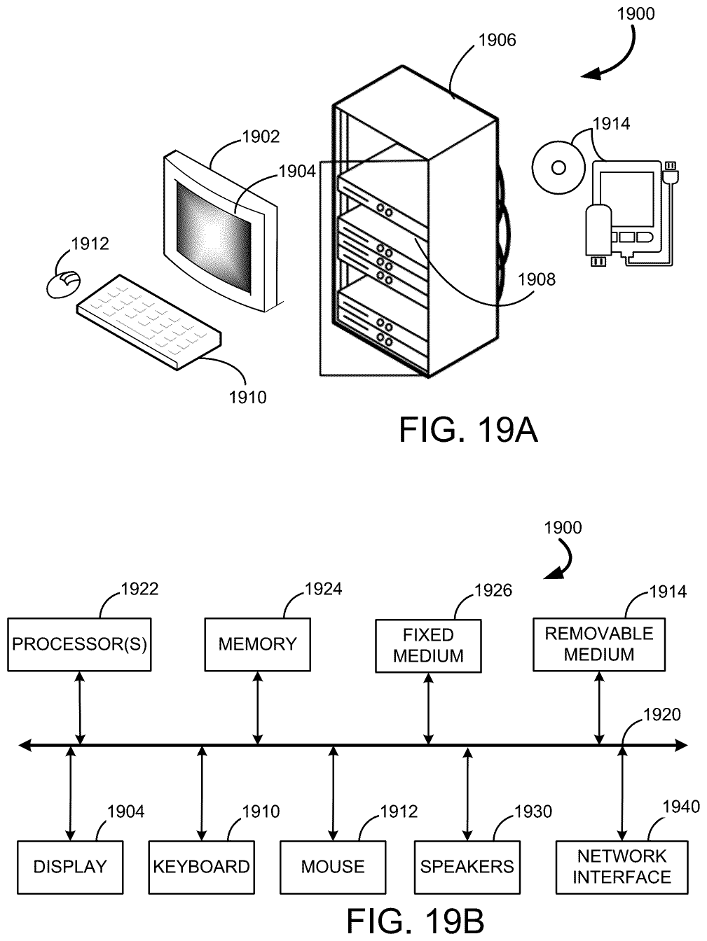

Now that the systems and methods for data normalization, algorithm certification, report generation and workflow approvals have been provided, attention shall now be focused upon apparatuses capable of executing the above functions in real-time. To facilitate this discussion, FIGS. 19A and 19B illustrate a Computer System 1900, which is suitable for implementing embodiments of the present invention. FIG. 19A shows one possible physical form of the Computer System 1900. Of course, the Computer System 1900 may have many physical forms ranging from a printed circuit board, an integrated circuit, and a small handheld device up to a huge supercomputer. Computer system 1900 may include a Monitor 1902, a Display 1904, a Housing 1906, server blades including one or more storage Drives 1908, a Keyboard 1910, and a Mouse 1912. Medium 1914 is a computer-readable medium used to transfer data to and from Computer System 1900. FIG. 19B is an example of a block diagram for Computer System 1900. Attached to System Bus 1920 are a wide variety of subsystems. Processor(s) 1922 (also referred to as central processing units, or CPUs) are coupled to storage devices, including Memory 1924. Memory 1924 includes random access memory (RAM) and read-only memory (ROM). As is well known in the art, ROM acts to transfer data and instructions uni-directionally to the CPU and RAM is used typically to transfer data and instructions in a bi-directional manner. Both of these types of memories may include any suitable form of the computer-readable media described below. A Fixed Medium 1926 may also be coupled bi-directionally to the Processor 1922; it provides additional data storage capacity and may also include any of the computer-readable media described below. Fixed Medium 1926 may be used to store programs, data, and the like and is typically a secondary storage medium (such as a hard disk) that is slower than primary storage. It will be appreciated that the information retained within Fixed Medium 1926 may, in appropriate cases, be incorporated in standard fashion as virtual memory in Memory 1924. Removable Medium 1914 may take the form of any of the computer-readable media described below.

Processor 1922 is also coupled to a variety of input/output devices, such as Display 1904, Keyboard 1910, Mouse 1912 and Speakers 1930. In general, an input/output device may be any of: video displays, track balls, mice, keyboards, microphones, touch-sensitive displays, transducer card readers, magnetic or paper tape readers, tablets, styluses, voice or handwriting recognizers, biometrics readers, motion sensors, brain wave readers, or other computers. Processor 1922 optionally may be coupled to another computer or telecommunications network using Network Interface 1940. With such a Network Interface 1940, it is contemplated that the Processor 1922 might receive information from the network, or might output information to the network in the course of performing the above-described confidential computing processing of protected information, for example PHI. Furthermore, method embodiments of the present invention may execute solely upon Processor 1922 or may execute over a network such as the Internet in conjunction with a remote CPU that shares a portion of the processing.

Software is typically stored in the non-volatile memory and/or the drive unit. Indeed, for large programs, it may not even be possible to store the entire program in the memory. Nevertheless, it should be understood that for software to run, if necessary, it is moved to a computer readable location appropriate for processing, and for illustrative purposes, that location is referred to as the memory in this disclosure. Even when software is moved to the memory for execution, the processor will typically make use of hardware registers to store values associated with the software, and local cache that, ideally, serves to speed up execution. As used herein, a software program is assumed to be stored at any known or convenient location (from non-volatile storage to hardware registers) when the software program is referred to as “implemented in a computer-readable medium.” A processor is considered to be “configured to execute a program” when at least one value associated with the program is stored in a register readable by the processor.

In operation, the computer system 1900 can be controlled by operating system software that includes a file management system, such as a medium operating system. One example of operating system software with associated file management system software is the family of operating systems known as Windows® from Microsoft Corporation of Redmond, Washington, and their associated file management systems. Another example of operating system software with its associated file management system software is the Linux operating system and its associated file management system. The file management system is typically stored in the non-volatile memory and/or drive unit and causes the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is, here and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

The algorithms and displays presented herein are not inherently related to any particular computer or other apparatus. Various general-purpose systems may be used with programs in accordance with the teachings herein, or it may prove convenient to construct more specialized apparatus to perform the methods of some embodiments. The required structure for a variety of these systems will appear from the description below. In addition, the techniques are not described with reference to any particular programming language, and various embodiments may, thus, be implemented using a variety of programming languages.

In alternative embodiments, the machine operates as a standalone device or may be connected (e.g., networked) to other machines. In a networked deployment, the machine may operate in the capacity of a server or a client machine in a client-server network environment or as a peer machine in a peer-to-peer (or distributed) network environment.

The machine may be a server computer, a client computer, a personal computer (PC), a tablet PC, a laptop computer, a set-top box (STB), a personal digital assistant (PDA), a cellular telephone, an iPhone, a Blackberry, Glasses with a processor, Headphones with a processor, Virtual Reality devices, a processor, distributed processors working together, a telephone, a web appliance, a network router, switch or bridge, or any machine capable of executing a set of instructions (sequential or otherwise) that specify actions to be taken by that machine.

While the machine-readable medium or machine-readable storage medium is shown in an exemplary embodiment to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding or carrying a set of instructions for execution by the machine and that cause the machine to perform any one or more of the methodologies of the presently disclosed technique and innovation.

In general, the routines executed to implement the embodiments of the disclosure may be implemented as part of an operating system or a specific application, component, program, object, module or sequence of instructions referred to as “computer programs.” The computer programs typically comprise one or more instructions set at various times in various memory and storage devices in a computer (or distributed across computers), and when read and executed by one or more processing units or processors in a computer (or across computers), cause the computer(s) to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually effect the distribution

While this invention has been described in terms of several embodiments, there are alterations, modifications, permutations, and substitute equivalents, which fall within the scope of this invention. Although sub-section titles have been provided to aid in the description of the invention, these titles are merely illustrative and are not intended to limit the scope of the present invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. In a zero-trust computing environment, a computerized method for normalizing runtime data for algorithm training, the method comprising:
projecting a first data set into a feature space using a transform model;
locally training the transform model to recognize and embed the identified features;
deploying the trained transform models at runtime sites, wherein each runtime site is in a sequestered computing node;
pre-processing a second data set using the transform model at each runtime site;
locally training the runtime model on the pre-processed data; and
performing federated training on a plurality of locally trained substantiations of the runtime model to generate a consolidated model.

2. The method of claim 1, further comprising identifying features within the feature space that impact the accuracy of a runtime model.

3. The method of claim 1, further comprising performing federated training on a plurality of the locally trained transform models.

4. The method of claim 1, wherein the first data is at least one image and the transform model is an image embedding model.

5. The method of claim 1, further comprising running the consolidated model on at least one additional dataset.

6. In a zero-trust computing environment, a computerized method for certification of algorithms and leakage detection of protected information, the method comprising:
within a certification environment:
receiving an algorithm and a known dataset;
processing the dataset using the algorithm to generate an output;
analyze the output against the known dataset to determine when the algorithm is performing data exfiltration;
flagging the algorithm when exfiltration is detected; and
locking the algorithm using a signature when no exfiltration is detected;
deploying the locked algorithm into deployment environments, wherein the deployment environments are substantiated within secure computing enclaves;
providing attestation of the locked algorithm; and
processing local datasets using the attested algorithm.

7. The method of claim 6, wherein the determining when the algorithm is performing data exfiltration includes one of a binary determination, risk as epsilon and threshold risk.

8. The method of claim 6, further comprising pre-certifying components of the algorithm in the certification environment.

9. The method of claim 8, locking combinations of pre-certified components of the algorithm.

10. The method of claim 6, wherein the certification environment is a secure computing environment.

11. The method of claim 6, wherein a detection model is trained when analyzing the output against the known dataset.

12. The method of claim 11, further comprising performing federated training of the detection model.

13. The method of claim 12, further comprising:
deploying the detection model at a plurality of data stewards;
processing algorithm outputs through the detection model; and
prohibiting the release of outputs that contain protected data.

14. The method of claim 13, wherein the detection model determines the permissions of the receiving party, and adjusts the type of data that is protected based on the permissions.

15. In a zero-trust computing environment, a computerized method for data processing using a foundational model, the method comprising:
receiving at least two inputs comprising a data specification, a data set and curated data set; and
applying a trained generative Artificial Intelligence (AI) model to the at least two inputs to generate an output, wherein:
when the inputs include the data specification and the data set, then generating the curated data set using the generative AI model;
when the inputs include the data set and the curated data, then generating the data specification using the generative AI model; and
when the inputs include the data specification and the curated data set, then generating a validation using the generative AI model.

16. In a zero-trust computing environment, a computerized method for report generation to guide algorithm usage, the method comprising:
calculating generalized tests error for the processing of an algorithm on a data set, wherein the processing is performed in a secure computing environment;
calculating a dynamic threshold, wherein the dynamic threshold is dependent upon the nature of protected information that is present in an output and the intended deployment of the output;
comparing the generalized tests error to the dynamic threshold to calculate a report;
provide the report to a data steward; and
determine whether or not to proceed with processing a data set of the data steward using the algorithm responsive to the report.

17. The method of claim 16, wherein the intended deployment is determined by the intended audience.

18. The method of claim 16, wherein different data types are provided different classifications, and wherein the nature of the protected information is responsive to these classifications.

19. The method of claim 18, wherein the data types include protected health information (PHI).

20. The method of claim 19, wherein the PHI is further subdivided by sensitivity levels.

21. The method of claim 20, wherein the report is at least one of a binary report and a risk gradient report.
